(19) United States
(12) Patent Application Publication
(10) Pub. No.: US 2024/0366472 A1
(43) Pub. Date: Nov. 7, 2024

(54) PACKAGING ARRANGEMENT FOR A BENEFICIAL AGENT

(71) Applicant: AKTIVAX, INC., Broomfield, CO (US)

(72) Inventors: Amir Genosar, Broomfield, CO (US); Greg Langley, Broomfield, CO (US); Rick Levaughn, Broomfield, CO (US); Andrew Fish, Broomfield, CO (US); Eric Sugano, Broomfield, CO (US); Erik Hoenes, Broomfield, CO (US); Dean Carpenter, Broomfield, CO (US); Dominic Aiello, Broomfield, CO (US); Justin Chatroop, Broomfield, CO (US); Nick Lucarelli, Broomfield, CO (US)

(21) Appl. No.: 18/687,517

(22) PCT Filed: Aug. 30, 2022
(86) PCT No.: PCT/US2022/075701

§ 371 Date: Feb. 28, 2024

Related U.S. Application Data
(60) Provisional application No. 63/238,943, filed on Aug. 31, 2021.

(51) Int. Cl. A61J 1/06; A61J 1/14; A61J 1/20

(52) U.S. Cl. CPC A61J 1/067; A61J 1/1406; A61J 1/2096

(57) ABSTRACT

A package arrangement is provided. The package arrangement includes a package including a compartment defined by a wall. The wall includes a flexible portion. The compartment stores a beneficial agent. The compartment at least partially collapses in response to applied pressure. The compartment is hermetically sealed in a storage configuration. The package includes a rigid insert including a conduit in fluid communication with the compartment. The rigid insert is positioned inside the compartment and includes an insert coupling portion. The package arrangement further includes a rigid frame containing part of the compartment within the rigid frame. The rigid frame includes a frame coupling portion. The insert coupling portion exerts pressure on the frame coupling portion through the flexible portion to restrict motion of the rigid insert relative to the rigid frame. The flexible portion hermetically seals a proximal portion of the conduit in the storage configuration.

### CROSS-REFERENCE TO RELATED APPLICATION(S)

The present application claims priority to U.S. Provisional Patent Application Ser. No. 63/238,943, filed Aug. 31, 2021, and entitled “Sealed unit dose package for storing and dispensing a beneficial agent,” which is incorporated by reference herein in its entirety. In the event that definitions or descriptive terms used in this or any other patent incorporated by reference conflict with definitions or descriptive terms used in the present application, the use in the present application will control.

### STATEMENT OF GOVERNMENT SUPPORT

This invention was made with Government support under contract no. HHSO100201800014C awarded by the Biomedical Advanced Research and Development Authority (BARDA) within the U.S. Department of Health and Human Services (HHS). The Government has certain rights in the invention.

### BACKGROUND

Beneficial agent administration devices are adapted to withdraw fluids from different types of containers, such as glass vials and plastic containers. Single-use containers are often provided to users as elements separate from the beneficial agent administration devices. Such arrangements can be cumbersome in environments where the beneficial agents need to be found and administered quickly. Single-use blister packs couplable with or integrated into single-use administration devices can be delicate and may not be suited for rougher administration environments, such as triage centers, because the single-use film containers (e.g., ones made from film, foil, or web materials) can be punctured or the layers of the single-use film containers can separate under stress.

### SUMMARY

The described technology provides a package arrangement. The package arrangement includes a package including a compartment defined by a wall. The wall includes a flexible portion. The compartment stores a beneficial agent. The compartment at least partially collapses in response to applied pressure. The compartment is hermetically sealed in a storage configuration. The package includes a rigid insert including a conduit (e.g., an internal conduit) in fluid communication with the compartment. The rigid insert is positioned inside the compartment and includes an insert coupling portion. The package arrangement further includes a rigid frame containing part of the compartment within the rigid frame. The rigid frame includes a frame coupling portion. The insert coupling portion exerts pressure on the frame coupling portion through the flexible portion to restrict motion of the rigid insert relative to the rigid frame. The flexible portion hermetically seals a proximal portion of the conduit in the storage configuration.

This summary is provided to introduce a selection of concepts in a simplified form that is further described below in the Detailed Description. This summary is not intended to identify key features or essential features of the claimed subject matter, nor is it intended to be used to limit the scope of the claimed subject matter.

Other implementations are also described and recited herein.

### BRIEF DESCRIPTIONS OF THE DRAWINGS

FIG. 1a illustrates a perspective view of an example of a package arrangement storing a liquid beneficial agent, showing an elongated rigid frame housing a package and a cap removably attached to the rigid frame.

FIG. 1b illustrates a perspective view of an example of a package.

FIG. 2a illustrates a longitudinal section of an example of a package arrangement for containing and dispensing a liquid beneficial agent in a first configuration.

FIG. 2b illustrates a longitudinal section of an example of a package arrangement in a second configuration.

FIG. 2c illustrates a cross-section of the package arrangement at a plane situated between the insert and the concave indentation.

FIG. 3a illustrates a longitudinal section of an example of a package arrangement in the second configuration wherein the fluid transport device is interfaced with a delivery device.

FIG. 3b illustrates a longitudinal section of an example of a package arrangement in the second configuration, following dispensing of the beneficial agent.

FIG. 4 illustrates a cross-section view of a package arrangement wherein the long axis of a package and the long axis of a fluid transport device are perpendicular.

FIG. 5 illustrates an exploded view of a package arrangement configured for use with a closed transfer system.

FIG. 6a illustrates a longitudinal section of an example of a package arrangement configured to be penetrated substantially perpendicularly relative to a longitudinal length of the package arrangement.

FIG. 6b illustrates the package arrangement after the cannula has been moved from the first configuration to a second configuration, wherein a portion of the cannula resides in a first conduit.

FIG. 7a illustrates a perspective view of an example of a package arrangement adapted for use in an auto-injector device or a similar device.

FIG. 7b illustrates a perspective view of an example of a package arrangement prior to the fluid transfer device being coupled to the rigid frame with a septum therebetween.

FIG. 8 illustrates an exploded view of an example of a package arrangement, revealing a bilateral symmetry of a rigid frame of the package arrangement, as well as bilateral symmetry of the attachment device.

FIG. 9a illustrates a longitudinal section of an example of a package arrangement in a first configuration.

FIG. 9b illustrates the package arrangement in a second configuration after dispensing of the beneficial agent.

FIG. 10 illustrates a cross-section view of an example of a package including a first formed side and a second flat lid side joined therebetween to form a compartment.

FIG. 11 illustrates an example package including an insert that includes a flange and a piercing member rigidly extending from the flange and passing through the first wall.

FIG. 12 illustrates an example package that includes an insert that includes a flange and a bore.

FIG. 13 illustrates an example package arrangement, including a Luer connector assembly.

### DETAILED DESCRIPTIONS

Single-use blister packs couplable with or integrated into single-use administration devices can be delicate and may not be suited for rougher administration environments, such as triage centers, because the single-use film containers (e.g., ones made from film, foil, or web materials) can be punctured or the layers of the single-use packages (e.g., film containers) can separate under stress. Providing a rigid frame can mitigate the risk that the film containers puncture or separate in environments where the package arrangement undergoes considerable stress (e.g., military and/or triage settings). The manner in which the package is positioned and coupled within the rigid frame can contribute to the protection of the package in these settings. The configuration of the package itself can also contribute to the protection of the package.

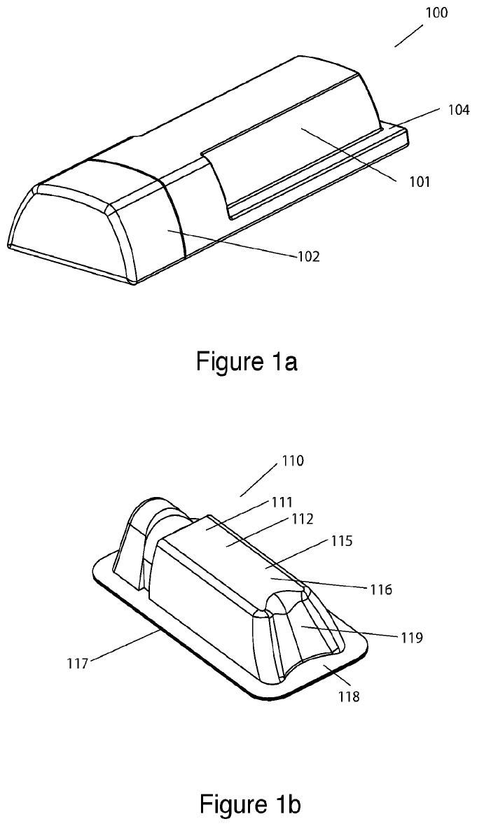

FIG. 1a illustrates a perspective view of an example of a package arrangement 100 storing a liquid beneficial agent, showing an elongated rigid frame 101 housing a package and a cap 102 removably attached to the rigid frame 101. The package may function as a container for a liquid beneficial agent to be administered to a patient. The rigid frame 101 can protect the package and a fluid transport device for accessing the package. The shape of the rigid frame 101 may approximately follow the contours of the package, including thin areas at the periphery of the package where web film walls of the package are joined together. A lip 104 of the rigid frame 101 disposed along a portion of the outer perimeter of the rigid frame 101 may at least partially enclose regions of the package where the web film walls are joined (e.g., a rim of the package), securing the package in place and limiting its movement. Removing the cap 102 may expose a fluid transport device, which can be manipulated to enable access to the beneficial agent contained in the package for dispensing and administration to a subject.

FIG. 1b illustrates a perspective view of an example of a package 110. The package 110 includes a compartment 111 for holding a liquid beneficial agent. The compartment may include a wall 112. The wall 112 may include an outer side 115 (e.g., an outer surface) and an inner side (e.g., an inner surface, not visible). The wall 112 may include a first wall 116, a second wall 117 and a rim 118, the rim 118 formed by joining the first wall 116 to the second wall 117 in a fluid-tight or hermetically sealed fashion along the respective perimeters of the first wall 116 and the second wall 117. The first wall 116 and the second wall 117 may be hermetically coupled to define a compartment 111 between the first wall 116 and the second wall 117. The compartment 111 may be adapted to contain a beneficial agent.

The rim 118 may at least partially define the coupling between the first wall 116 and the second wall 117. The rim 118 may be composed of material of one or more of the first wall 116 or the second wall 117. In some embodiments, the first wall 116 is preformed or molded to create a concave portion that may form the compartment 111 for containing a beneficial agent when the first wall 116 is joined to the second wall 117. That is, the second wall 117 may be substantially planar such that the compartment is formed by the concave portion of the first wall 116. The first wall 116 may also form components needed for interfacing with a fluid transport device. The first wall 116 may be additionally preformed to optimize the geometry of the package 110 for collapsing under pressure to maximize recovery of the beneficial agent from the package 110 during dispensing (i.e., to minimize the amount of leftover beneficial agent that cannot be extracted from the package 110) and to achieve predictable, consistent changes in shape of the package 110 as it collapses, in order to reduce or minimize variations between packages in their collapse dynamics. A concave indentation 119 in the first wall 116 may be disposed at an end of the package 110 opposite of a delivery end is an example of a geometric feature that can facilitate the collapsing of package 110 by initiating the collapse and leading the sequence of wall 112 movements. The wall 112 may also be formed to follow contours of an insert (not shown) disposed in the package 110 and may be configured to accept a frame coupling portion (e.g., a protrusion) on the interior of a rigid frame 101 that may secure the package 110 and may minimize movements thereof relative to the rigid frame 101. In an implementation, the package arrangement 100 may include a void between the package 110 and the rigid frame 101. The package arrangement 100 may further include a pressure modification element to control the pressure in the void, the controlled pressure adapted to facilitate the expulsion of the beneficial agent from the compartment 111. In an implementation, the pressure modification element includes a hole in the rigid frame 101 to allow air to enter the rigid frame 101 to facilitate the collapse of the compartment 111. In another implementation, the pressure modification element includes a fluid pressure element that introduces pressurized fluid into the void to facilitate the collapse of the compartment 111.

In an implementation, the rim 118 extends around the package at the coupling between the first wall 116 and the second wall 117. For example, in the illustrated implementation, the rim 118 is substantially rectangular (e.g., with curvilinear corners). In other implementations, the rim 118 may circumscribe fewer sides and/or a smaller portion (e.g., one side, two sides, three sides, a portion of the of a circumference, a portion of a perimeter, or the like) of the compartment 111 than the four sides illustrated.

In an implementation, the wall 112 includes a flexible portion. For example, the first wall 116 may be or include a flexible molded wall that at least partially defines the flexible portion. In implementations, the entire wall may be flexible. In an implementation, the flexible molded wall may include a cavity formed by molding the flexible molded wall. In an implementation, the second wall 117 is an opposing wall that is hermetically coupled with the flexible molded wall. The opposing wall may be composed of a rigid material, a flexible material, or a combination thereof. In an implementation, the flexible portion is composed of a flexible material, such as a film, a foil, or a webbing. In an implementation, a portion of the first wall 116 (e.g., a flexible molded wall) is formed to conform to an interior shape of the rigid frame.

A second wall 117 may be a lidding wall. A lidding wall may be approximately flat in shape, or it may be preformed to create a cavity. The package 110 may comprise a rim 118 where the first wall 116 and the second wall 117 are joined together in a fluid-tight fashion, in part defining the volume of the compartment 111. The rim 118 may be formed by heat-sealing, gluing, welding, or other methods known in the art or a combination thereof.

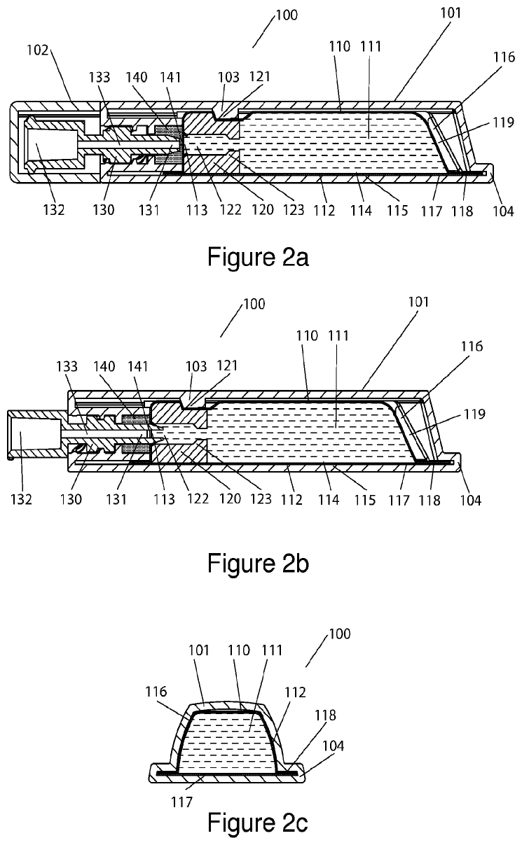

FIGS. 2a-2c illustrate cross-section views of a package arrangement 100 of FIGS. 1a-1b in storage and dispensing configurations. The package arrangement 100 illustrated in FIGS. 2a-2c may be an implementation of the package arrangement 100 illustrated in FIGS. 1a-1b, and like named and/or numbered elements may be implementations of one another. Specifically, FIG. 2a illustrates a longitudinal section of an example of a package arrangement 100 for containing and dispensing a liquid beneficial agent in a first configuration (e.g., a sealed configuration or a storage configuration). The illustrated example package arrangement includes a package 110, a fluid transport device 130, and a septum 140. The package 110 may include a compartment 111 for holding the beneficial agent (e.g., a fluid or a fluid and dried agent to be combined). The compartment 111 may include a wall 112. The wall 112 may include a communication region 113, an inner side 114 facing the interior of the compartment in contact with the beneficial agent, and an outer side 115 opposite of the inner side 114. The package 110 may further include a rigid insert 120 disposed inside of the compartment 111 that confronts the communication region 113 of the wall 112.

In an implementation, the wall 112 may include a first wall 116 (e.g., a flexible molded wall) with a cavity. The first wall 116 may at least partially define a flexible portion of the wall 112. In an implementation, the wall 112 may include a second wall (e.g., an opposing wall) hermetically coupled with the first wall 116 to seal the cavity and at least partially define the compartment 111 between the first wall 116 and the second wall 117.

In an implementation, the package arrangement 100 may include a package 110. The package 110 may include the compartment 111 defined by the wall 112. The wall 112 may include a flexible portion. The compartment 111 may be adapted to store a beneficial agent. The compartment 111 may be adapted to at least partially collapse in response to applied pressure. In an implementation, the compartment may be hermetically sealed in the first configuration.

In an implementation, the package 110 may further include a rigid insert 120. The rigid insert 120 may include a conduit 122 (e.g. an internal conduit), The conduit 122 may be in fluid communication with the compartment 111. In an implementation, the rigid insert 120 may be positioned inside the compartment 111. The rigid insert 120 may include an insert coupling portion 121 (e.g., a notch, divot, a groove, a protrusion, or a recess)). The conduit 122 may be positioned on an opposite side of the wall 112 relative to an access region 141 of the septum 140. The access region 141 may enable fluid communication between a passageway 133 of the fluid transport device 130 and an interior of the compartment 111. A communication region 113 of the wall 112 may be disposed between the access region 141 and the conduit 122.

In an implementation, the communication region 113 of the wall 112 may be configured to be pierced by the fluid transport device 130. The piercing may transition the package arrangement 100 from a first configuration (e.g., a sealed configuration or a storage configuration) in which the communication region 113 prevents fluid communication between the passageway 133 and the compartment 111 to a second configuration (e.g., a pierced configuration, a dispensing configuration, a ruptured configuration, or a delivery configuration) in which the communication region 113 is ruptured by the piercing. The rupture may enable fluid communication between the passageway 133 and the compartment 111 via the conduit 122. In an implementation, the access region 141 may be pierceable by the fluid transport device 130 during the transition. The access region 141 may be positioned to interface with the communication region 113. The septum 140 may be positioned and/or compressed between the package 110 and the fluid transport device 130.

In an implementation, the conduit 122 includes a wide portion with a width wider than a width of the fluid transport device 130 (e.g., a width of the fluid transport device 130 adapted to be introducible to the conduit 122) and a narrow portion with a width narrower than the width of the fluid transport device 130, the narrowed width of the narrow portion restricting the extent to which the fluid transport device can penetrate the conduit 122.

The package arrangement 100 may include a rigid frame 101. The rigid frame 101 may contain at least a portion (e.g., may contain a portion or all) of the compartment within the rigid frame 101. The rigid frame 101 may include a frame coupling portion 103 (e.g., a protrusion, recess, or notch). The insert coupling portion 121 is configured to exert pressure on the frame coupling portion 103 through the wall 112 (e.g., a flexible portion of the wall) to restrict motion of the rigid insert 120 relative to the rigid frame 101. The wall (e.g., a flexible portion of the wall) hermetically seals a proximal portion of the conduit 122 in the first configuration.

In an implementation, the insert coupling portion 121 may include one of a recess and a protrusion, and the frame coupling portion 103 may include the other of the recess and the protrusion to couple with the insert coupling portion. The rigid insert 120 may include a proximal portion located proximally relative to the insert coupling portion 121. The proximal portion may have a transverse cross-section of a size different from a size of a transverse cross-section of the insert coupling portion 121. In an implementation, the protrusion may protrude substantially orthogonally to a longitudinal length of the rigid frame 101. As used herein, the longitudinal length means the length of the longest side of the package arrangement. As used herein, a direction of a longitudinal length or a longitudinal axis may be substituted with a direction in which the fluid transport device 130 penetrates the rigid insert 120.

In an implementation, the hermetic coupling between the first wall 116 and the second wall 117 may at least partially define the rim 118. The rim 118 may extend away from the compartment 111. The rigid frame 101 may further include a lip 104 with a hollow groove in the interior of the rigid frame. In an implementation, at least a portion of the rim 118 may be positioned within the hollow groove. In an implementation, the hollow groove may exert pressure on the rim 118 to reinforce the coupling between the first wall 116 (e.g., a flexible molded wall) and the second wall 117 (e.g., an opposing wall).

The rigid insert 120 may be configured to interface the package 110 with the fluid transport device 130 and/or with features in the rigid frame 101 to direct the fluid transport device 130 into the compartment 111, while limiting movements of the package 110 relative to the fluid transport device 130 and the rigid frame 101. For example, the rigid insert 120 may comprise an insert coupling portion 121 (e.g., a notch, divot, a groove, a protrusion, receptacle, or a recess) configured to accept and mate to a frame coupling portion 103 (e.g., a notch, divot, a groove, a protrusion, or a recess that complements the insert coupling portion 121 for coupling) on the interior of the rigid frame 101. The rigid insert 120 may also comprise a receptacle 123 shaped to accept a portion of the fluid transport device 130 and to form a fluid-tight seal between the contact surfaces of the receptacle and the fluid transport device 130 (e.g., after the septum is penetrated).

In some embodiments, the lip 104 may include a hollow groove configured to accept the rim 118 of the package 110 to secure said package 110 in place and to reduce the possibility of the rim 118 losing integrity as a result of the first wall 116 and the second wall 117 separating if the beneficial agent is accidentally pressurized. Specifically, withdrawing beneficial agents from rigid containers such as glass vials with a syringe may require users to first inject air into the container to compensate for the empty volume created as a result of the beneficial agent being removed from the container. Without first injecting air into the container, gas pressure inside the container may fall below atmospheric pressure when a beneficial agent is withdrawn, causing ambient air to exert a force on the syringe plunger opposite of the force exerted by the user. Even though injecting air into flexible containers may be unnecessary as the flexible containers collapse under ambient atmospheric pressure when a beneficial agent is withdrawn, users accustomed to glass vials may accidentally inject air into the package 110 due to habit. The resulting pressure increase in the compartment 111 may cause the beneficial agent and introduced air to exert a peeling force on the rim 118, acting to separate the first wall 116 from the second wall 117. If the first wall 116 and the second wall 117 become separated, the fluid-tight seal will fail, compromising the integrity of the package 110. In these configurations, the lip 104, therefore, may reduce the risk of seal failure by pinching the rim 118 to restrict the first wall 116 from peeling away from the second wall 117.

FIG. 2a additionally illustrates the fluid transport device 130, including a distal end 131 for accessing the beneficial agent contained in the compartment 111, a proximal end 132 for dispensing the beneficial agent, and a passageway 133 disposed between the distal end 131 and the proximal end 132. The fluid transport device 130 may be configured at its proximal end 132 to interface with a delivery device such as a syringe, for example, by a Luer-type connector. The fluid transport device 130 may comprise an integrated delivery device such as a needle. In some embodiments where the package arrangement 100 serves as a component of a prefilled drug delivery system, the fluid transport device 130 may interface with a delivery device, an administration device, or a connector thereto.

In some embodiments of the package arrangement 100, the package 110 may comprise multiple compartments that can be merged by breaking a frangible seal between the compartments as taught by U.S. Pat. No. 9,820,913, which is incorporated herein by reference in its entirety. In the event that definitions or descriptive terms used in this or any other patent incorporated by reference conflict with definitions or descriptive terms used in the present application, the use in the present application will control. In some implementations, the rigid insert 120 and the beneficial agent may be disposed in two isolated compartments separated by a frangible seal such that the rigid insert 120 does not come in contact with the beneficial agent until the frangible seal is opened, as taught by U.S. Pat. No. 9,820,913.

The septum 140 disposed on the outer side 115 of the wall 112 of the package 110 may confront the communication region 113 of the wall 112 (e.g., a flexible wall or a wall with a flexible portion) and may include the access region 141 configured to direct the fluid transport device 130 into the package 110, wherein the septum 140 establishes a fluid-tight seal that restricts the flow of the beneficial agent from the compartment 111 into the passageway 133 of the fluid transport device 130 and reduces or eliminates flow of any fluid surrounding the package 110 into said package 110 and into said passageway 133. In other words, the contact surfaces at the interfaces of the fluid transport device 130 and the septum 140, and the access region 141 of the septum 140 and the communication region 113, are fluid-tight in a first configuration (e.g., a sealed configuration or a storage configuration). In some embodiments of the package arrangement 100, the properties of the wall 112 are such that it can form a fluid-tight seal around the fluid transport device 130 without requiring a septum 140, and, as such, the septum 140 can be eliminated.

The fluid transport device 130 is moveable between a first configuration (e.g., a sealed configuration or storage configuration) and a second configuration (e.g., a pierced configuration, a dispensing configuration, a ruptured configuration, or a delivery configuration). In the first configuration shown in FIG. 2a, the communication region 113 of the wall 112 and the access region 141 of the septum 140 are not compromised, and the beneficial agent is sealed inside the package 110 in a fluid-tight fashion. To transition to the second configuration, the distal end 131 pierces through the access region 141 and the communication region 113 to establish fluid communication between the compartment 111 and the passageway 133. In implementations, the transition from the first configuration to the second configuration may be accomplished by simply pushing the fluid transport device 130 toward the package 110 or by screwing the fluid transport device 130 into the package 110. Some embodiments of the package arrangement 100 may further include biasing elements such as springs and/or snap-fit elements to bias the fluid transport device 130 to a configuration the fluid transport device is in (i.e., when in the first configuration, the fluid transport device is biased towards that configuration, but once a user transitions the fluid transport device to the second configuration, the biasing elements begin biasing the fluid transport device to the second configuration). This type of arrangement can lower the risk of accidentally dispensing the beneficial agent and ensures that the transition between the first and the second configurations is rapid and complete. In some implementations, the transition may be irreversible.

A package arrangement 100 may include a rigid frame 101 enclosing the package 110 and a cap 102 removably attached to the rigid frame 101. The cap 102 may surround, or otherwise separate from the external environment, the proximal end 132 of a fluid transport device 130. This may ensure that the surfaces of the fluid transport device 130 that come in contact with the beneficial agent during dispensing remain sterile during storage.

In some embodiments, the shape of the concave indentation 119 may not follow the shape of the rigid frame 101 and may not be in complete contact with an interior side of the rigid frame 101. The unoccupied space between the concave indentation and the portion of the rigid frame 101 opposed to the concave indentation enables the wall 112 to expand into said space when deformed, for example, when pressure is applied at the communication region 113 by the fluid transport device 130 before the region is pierced.

FIG. 2b illustrates a longitudinal section of an example of a package arrangement 100 in the second configuration. The fluid transport device 130 has been moved towards the package 110, and the proximal end 132 has pierced through the access region 141 of the septum 140 and the communication region 113 of the wall 112, establishing fluid communication between the compartment 111 and the passageway 133 of the fluid transport device. In the second configuration, the beneficial agent contained in the compartment 111 can be dispensed (e.g., withdrawn into a delivery device, such as a syringe or injector) through the distal end 131 of the fluid transport device 130.

FIG. 2c illustrates a cross-section of the package arrangement 100 at a plane situated between the rigid insert 120 and the concave indentation 119. As can be seen in the Figure, the shape of the wall 112 of the package 110 closely follows the interior contours of the rigid frame 101. A groove in the lip 104 encloses the rim 118 at a position where the first wall 116 and the second wall 117 are joined. The rigid frame 101, therefore, provides structural support to the package to reduce the risk of loss of integrity of the fluid-tight seal at the interface of the first and the second wall and within the wall itself. However, the top surface of the first wall 116 that is parallel to the second wall 117 may not follow a shape of the rigid frame 101. A gap may be present between said portion of the first wall 116 and the rigid frame 101, allowing for a momentary increase in the volume of the compartment 111 and the deformation of the first wall 116 when a fluid transport device transitions from the first configuration to the second configuration, allowing for a deformation to take place in a controlled manner.

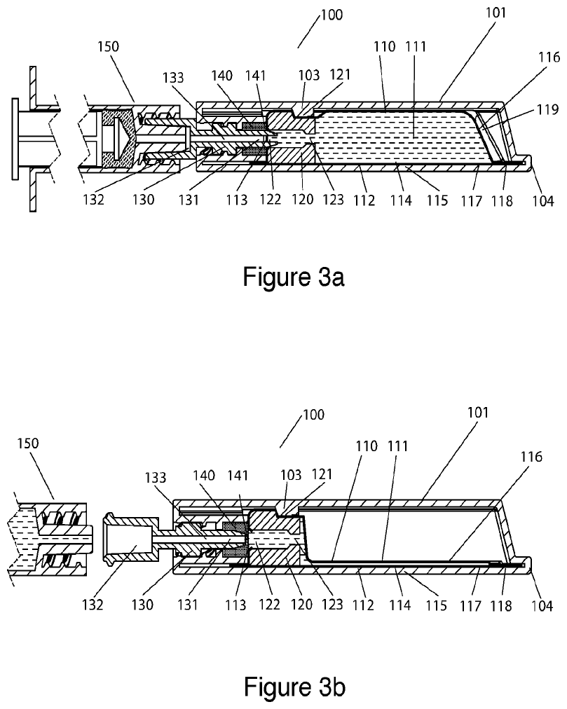

FIGS. 3a-3b illustrate cross-section views of a package arrangement with a delivery device. The package arrangement 300 may recite features similar to the package arrangement 100 illustrated in FIGS. 1a-1b and 2a-2c, and like-numbered and/or like-named features of the package arrangement 300 may be arranged and may function similarly to the features of the package arrangement 100, except as otherwise specified. Specifically, FIG. 3a illustrates a longitudinal section of an example of a package arrangement 100 in the second configuration wherein the fluid transport device 130 is interfaced with a delivery device 150. In the pictured embodiment, the delivery device 150 is a syringe comprising a Luer-lock fitment that has been screwed onto the proximal end 132 of the fluid transport device 130, establishing fluid communication between the compartment 111, the passageway 133, and a reservoir of the delivery device 150.

FIG. 3b illustrates a longitudinal section of an example of a package arrangement 100 in the second configuration, following dispensing of the beneficial agent. The package 110 may collapse due to negative pressure exerted by the delivery device 150, which may deform the first wall 116 and reduced the volume of the compartment 111. In the illustrated implementation, the delivery device 150 has been disengaged from the proximal end 132 of the fluid transport device 130, and the fluid transport device has been retracted to the first configuration. Although the communication region 113 of the wall 112 may have been irreversibly compromised, as the wall 112 may be made from thin web material, the access region 141 of the septum 140 may reseal. In the illustrated implementation, the septum 140 is constructed of a compliant elastomeric material, and the access region 141 is prescored for controlled rupturing. This arrangement can enable resealing of the package arrangement after dispensing the beneficial agent, which can further enable multi-dose presentations and can reduce potential spills of beneficial agents that may be cytotoxic, radioactive, and/or highly potent.

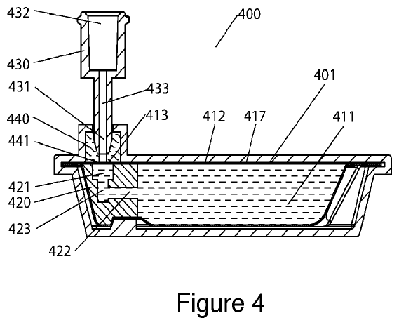

FIG. 4 illustrates a longitudinal section of an example of a package arrangement 400 wherein the passageway 433 of the fluid transport device 430 connects its proximal end 432 and distal end 431 and is arranged generally in perpendicularly to a direction of a longitudinal axis of the package 401. The package arrangement 400 may recite features similar to the package arrangement 100 illustrated in FIGS. 1a-1b and 2a-2c, and like-numbered and/or like-named features of the package arrangement 400 may be arranged and may function similarly to the features of the package arrangement 100, except as otherwise specified.

In an implementation, the fluid transport device 430 may be configured to pierce a flexible portion of a wall 412. The piercing may cause the package arrangement 400 to transition from a first configuration (e.g., a sealed configuration or a storage configuration) in which a compartment 411 is hermetically sealed from a passageway 433 to a second configuration (e.g., a pierced configuration, a dispensing configuration, a ruptured configuration, or a delivery configuration) in which the compartment 411 is in fluid communication with the passageway 433. The piercing may be in a direction at least partially orthogonal to a longitudinal length of the package arrangement 100. The pierced flexible portion may be positioned on a side of the package arrangement 400 opposite a side of the package arrangement 400, including an insert coupling portion. In this implementation, the conduit 423 may include a substantially right angular bend defined by a first portion 422 and a second portion 421. The first portion 422 of the conduit 423 may be substantially parallel with a longitudinal length of the package arrangement 400. The first portion 422 may be in direct fluid communication with the compartment 411. The second portion 421 of the conduit 423 may be substantially orthogonal to the longitudinal length. The second portion 421 may be in indirect fluid communication with the compartment 411. The second portion 421 may be configured to receive at least a portion of the fluid transport device 430.

In the illustrated implementation, the insert 420 may include a conduit 423. The conduit 423 may include a first portion 422 and a second portion 421 that communicates with the first portion 422 and the compartment 411. The communication region 413 may include a portion of the second wall 417 of the wall 412. The fluid transport device 430 may translate between a first configuration (e.g., a storage configuration or a sealed configuration) and a second configuration (e.g., a pierced configuration, a dispensing configuration, a ruptured configuration, or a delivery configuration). In the configuration of FIG. 4, the communication region 413 of the wall 412 and the access region 441 of the septum 440 are not compromised, and the fluid beneficial agent is sealed inside the compartment 411. The device may comprise a septum 440 comprising an access region 441 confronting the communication region 413. In the second configuration these are compromised by the distal end 431 of the fluid transport device 430, and the distal end 431 pierces through the access region and the communication region to establish fluid communication between the compartment 411 and the passageway 433.

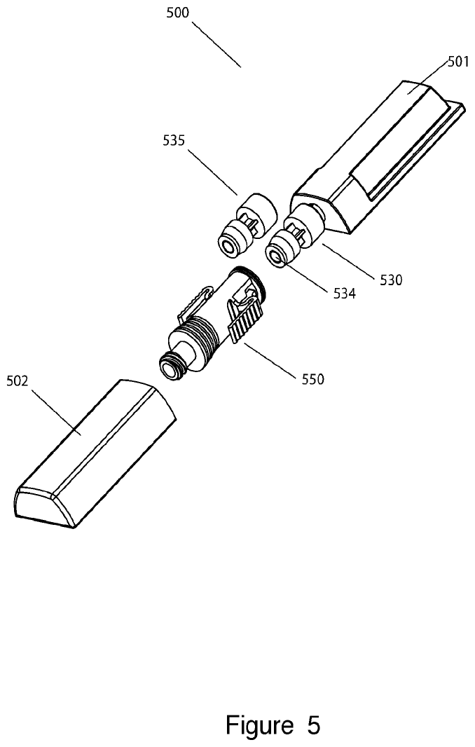

FIG. 5 illustrates a perspective view of an example of a package arrangement 500 adapted for use with a closed transfer system. The package arrangement 500 comprises a package enclosed in a rigid frame 501 containing a beneficial agent, a fluid transport device 530, a Luer-lock adaptor 535, a syringe adaptor 550, and a cap 502. In the illustrated implementation, the fluid transport device 530 may include a Luer-lock fitment comprising a sealing septum 534 to prevent leakage of the beneficial agent. The Luer-lock adaptor 535 is similarly equipped with a sealing septum and is intended to be installed on an IV line. The syringe adaptor 550 connects to the fluid transport device 530 on a first end and to a syringe on a second end. Once the beneficial agent is transferred to the syringe, the user connects the syringe adaptor 550 to the Luer-lock adaptor 535 to transfer the beneficial agent to the IV line.

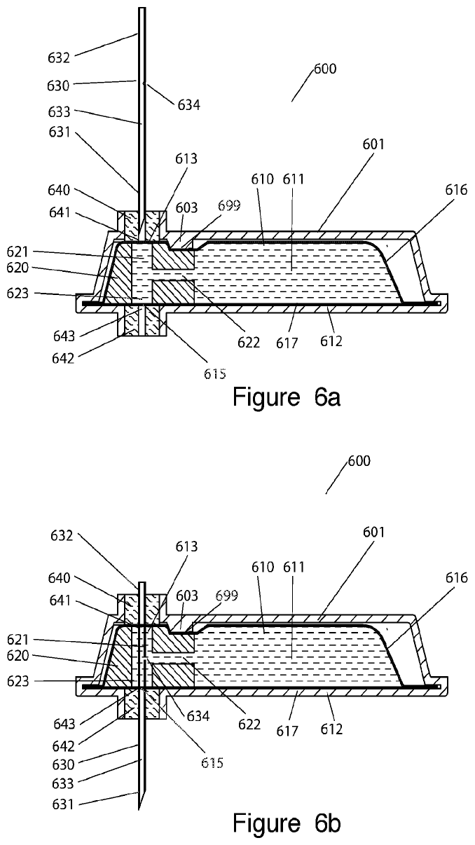

FIGS. 6a-6b illustrate a cross-section of an example package arrangement 600 wherein a fluid transport device comprises a fluid transport device (e.g., in the form of a cannula). The package arrangement 600 may recite features similar to the package arrangement 100, and like-numbered and/or like-named features of the package arrangement 600 may be arranged and may function similarly to the features of the package arrangement 100, except as otherwise specified.

Specifically, FIG. 6a illustrates a longitudinal section of an example of a package arrangement 600 configured to be penetrated substantially perpendicularly relative to a longitudinal length of the package arrangement 600. The package arrangement may include a package 610, a fluid transport device 630, a first septum 640, a second septum 642, and a rigid frame 601. The first septum 640 may include a first access region 641. The second septum 642 may include a second access region 643.

In an implementation, the package arrangement 600 includes a fluid transport device 630 (e.g., a cannula, as illustrated, or a needle). The fluid transport device 630 may include a passageway 633, a proximal end 632 with a first opening in fluid communication with the passageway 633, a distal end 631 with a second opening in fluid communication with the passageway 633, and/or a hole 634 in a sidewall of the fluid transport device 630 between the proximal end 632 and the distal end 631. The hole 634 in the sidewall may be in fluid communication with the passageway 633. In an implementation, a conduit 623 may include a first portion 622 substantially parallel to a longitudinal length of the package arrangement 600. The first portion 622 may be in direct fluid communication with the compartment 611. The conduit 623 may further include a second portion 621 substantially orthogonal to one or more of the longitudinal length of the package arrangement 600 or a longitudinal length of the first portion 622. The second portion 621 may be in indirect fluid communication with the compartment 611 via the first portion 622. The second portion 621 may be hermetically sealed by the wall 612 on a first end at a first communication region 613 of the second portion 621 and on a second end of the second portion 621 at a second communication region 615 in a first configuration. In an implementation, the fluid transport device 630 is configured to transition the package arrangement 600 from a first configuration (e.g., a sealed configuration or a storage configuration) in which the compartment 611 is hermetically sealed from the passageway 633 to a second configuration (e.g., a pierced configuration, a dispensing configuration, a ruptured configuration, or a delivery configuration) in which the compartment 611 is in fluid communication with the passageway. The fluid transport device 630 may cause the transition by the piercing the wall 612 at the first end at the first communication region 613, advancing through the second portion 621, and piercing the wall 612 at the second end at the second communication region 615. The transition may establish fluid communication between the passageway 633 and the conduit 623 via the hole 634 in the sidewall of the fluid transport device 630. In an implementation, the pierced first communication region 613 and the pierced second communication region 615 maintain a hermetic seal about the fluid transport device 630 in the second configuration (e.g., a pierced configuration, a dispensing configuration, a ruptured configuration, or a delivery configuration). In an implementation, the fluid transport device 630 may be of a shape (e.g., straight, as illustrated) such that the arrangement of the first portion 622 relative to the second portion 621 prevents the fluid transport device 630 from entering the first portion 622 if the fluid transport device 630 pierces both the first communication region 613 and the second communication region 615.

The package 610 comprises a wall 612 comprising a first wall 616 and a second wall 617, peripherally sealed to each other to form a compartment 611 containing a beneficial agent. The package further comprises an insert 620 disposed in the compartment 611 in fluid communication with the beneficial agent. The rigid frame 601 of the package arrangement 600 contains the package 610 and mechanically restrains the insert 620 from moving. The rigid frame 601 may comprise a frame coupling portion 603 configured to mate with an insert coupling portion 699 of the insert 620.

The first septum 640 confronts the first wall 616 of the package 610 and is restrained by the rigid frame 601 such that it is compressed against the first wall 616 to form a fluid-tight seal between the first septum 640 and the first wall 616. The second septum 642 confronts the second wall 617 and is restrained by the rigid frame 601 such that it is compressed against the second wall 617 to form a fluid-tight seal between the second wall 617 and the second septum 642.

The insert 620 comprises a conduit 623 aligned with the first septum 640 and the second septum 642, configured to receive the fluid transport device 630, and a second portion 621 communicating between the first portion 622 and the compartment 611.

In the first configuration, the fluid transport device 630 may confront the first wall 616. The fluid transport device 630 comprises a proximal end 632, a proximal end 632, a passageway 633, and a hole 634 in a sidewall between the proximal end and the distal end in fluid communication with the passageway 633. As will be illustrated in FIG. 6b, the fluid transport device 630 serves as an administration device for delivering the beneficial agent to a desired target, such as the intramuscular, subcutaneous, or intradermal space of a subject or topically to a subject. The fluid transport device 630 can be made from a variety of materials and structures, such as stainless steel or Teflon.

FIG. 6b illustrates the package arrangement 600 after the fluid transport device 630 has been moved from the first configuration to a second configuration, wherein a portion of the fluid transport device 630 resides in a first portion 622. The first wall 616 and the second wall 617 are punctured by the fluid transport device, and the first septum 640 and the second septum 642 prevent the beneficial agent from escaping between the fluid transport device 630 and the first wall 616 and between the fluid transport device 630 and the second wall 617. The hole 634 of the fluid transport device 630 establishes fluid communication between the compartment 611 and the proximal end 632 of the fluid transport device 630. In some configurations, the proximal end 632 of the fluid transport device 630 is blocked. In some configurations, in particular, where the fluid transport device 630 is made from a soft material, a guide needle may be accommodated in the fluid transport device 630, for example, to give it structural rigidity and facilitate its penetration of the first wall 616 and/or the second wall 617, and/or the tissue of the subject as it moves from the first configuration to the second configuration. After the fluid transport device 630 is moved from the first configuration to the second configuration, the guide needle can be fully or partially withdrawn through the proximal end 632 of the fluid transport device 630.

In one example of the package arrangement 600, pressurized fluid may be introduced into a void between the package 610 and the rigid frame 601 to urge the beneficial agent to expel through fluid transport device 630. In another example, the package arrangement 600 further comprises a pump for advancing the beneficial agent to the proximal end 632 of the fluid transport device 630, and at least a portion of the pump may reside in the insert 620. In yet another example, the package arrangement 600 further comprises a flow sensor for measuring the advancement of the beneficial agent to the proximal end 632 of the fluid transport device 630. At least a portion of the flow sensor may reside in the insert 620.

In some embodiments of the package arrangement 600, the properties of the first wall 616 and/or the second wall 617 are such that said walls can form a fluid-tight seal around the fluid transport device 630 without requiring a septum, and, as such, the septum can be eliminated.

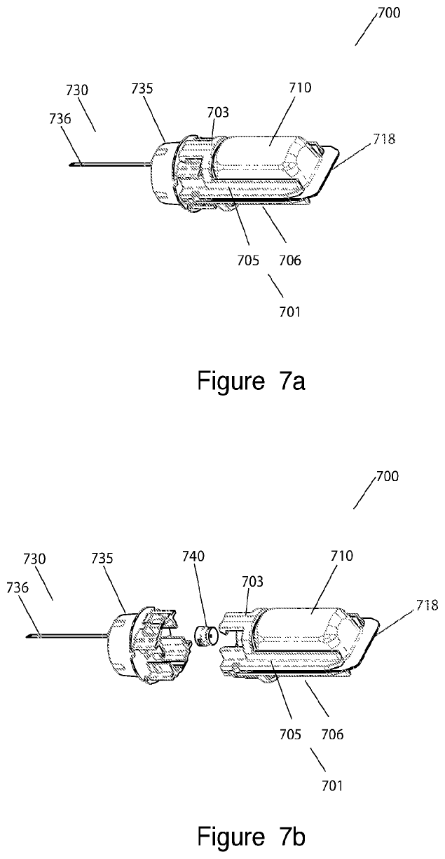

FIGS. 7a-7b, 8, and 9a-9b illustrate an implementation of a package arrangement 700 with a corresponding delivery device system. The package arrangement 700 may be adapted for use in an auto-injector device or a similar device. While not described in detail herein, the auto-injector device may be provided according to any of the teachings of U.S. Pat. No. 10,716,901 and/or U.S. patent application Ser. No. 16/933,779, which are incorporated by reference herein in their entirety. The package arrangement 700 may include a rigid frame 701, comprising a first clip 705 and a second clip 706, with the package 710 disposed therebetween. The first clip 705 and the second clip 706 comprise interlocking cantilever snap-fit features, enabling snap-fit assembly of the clips into the rigid frame 701. A frame coupling portion 703 of the first clip 705 secures the package 710 in the rigid frame 701. The fluid transport device 730 of the package arrangement 700 comprises an attachment device 735, an administration device 736 (e.g., a needle or other element for injection or introduction), and a hub (obscured in the figure). The package arrangement 700 additionally comprises an attachment device 735, comprising cantilever snap-fit features for coupling the attachment device 735 with the rigid frame 701.

FIG. 7b illustrates exploded views of an example package arrangement configured for use in an auto-injector device. FIG. 7a illustrates a perspective view of an example of a package arrangement 700 adapted for use in an auto-injector device or a similar device. FIG. 7b illustrates a perspective view of an example of a package arrangement 700, prior to the fluid transport device 730 being coupled to the rigid frame 701 with a septum 740 therebetween. Prior to the fluid transport device 730 being coupled to the rigid frame 701 with a septum 740 therebetween, a proximal end of the first clip 705 includes a first clip end configured to mate with the attachment fluid transport subassembly including the fluid transport device 730. A distal rail protrusion of the first clip 705 secures the package 710, as shown. A package insert may be configured to set the package 710 fast within the rigid frame 701 subassembly. Coupling of the attachment device 735 to the rigid frame 701 may compress the septum 740 against a communication region of the package 710, creating a fluid-tight seal at the contact surfaces between the septum 740 and the package 710. For example, the attachment device 735 may be couplable to an assembly formed by the coupling of the first clip 705 and the second clip 706. The attachment device 735 may be configured to fix the septum 740 to the wall 712 and orient an access region 741 of the septum 740 configured to be pierced by the fluid transport device 730 to coincide with an opening 722 of the rigid insert 720 on opposite sides of the wall 712 in a first configuration (e.g., a storage configuration or a sealed configuration).

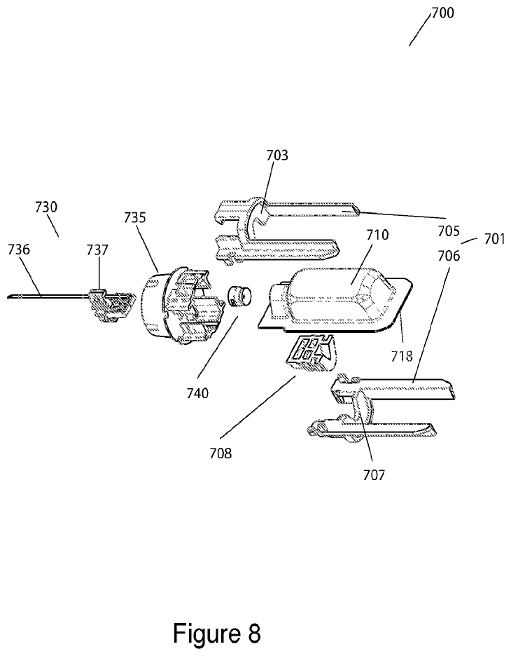

FIG. 8 illustrates an exploded view of an example of a package arrangement 700, revealing a bilateral symmetry of a rigid frame 701 of the package arrangement 700, as well as bilateral symmetry of the attachment device 735, which enable the package arrangement 700 to be configured with two packages containing beneficial agents, two fluid transfer devices, and two septa. The implementation of the package arrangement 700 illustrated in FIG. 8 is configured for use with a single package of the beneficial agent and additionally comprises a null fitment 708 used in place of a second package. The package 710 may include a rigid insert 720 configured to hold fast the package 710 between the distal rails of the rigid frame 701. An insert coupling portion 723 (e.g., a notch, divot, a groove, a protrusion, or a recess) in the package 710 is configured to accept a frame coupling portion 703 (e.g., a notch, divot, a groove, a protrusion, or a recess that complements the insert coupling portion 723 for coupling) of the first clip 705.

In an implementation, the first clip 705 may be configured to engage a first portion of the wall 712. The first clip 705 may include the frame coupling portion 703, and the first portion of the wall 712 may include the insert coupling portion 723. The second clip 706 may be configured to engage a second portion of the wall 712. The second portion of the wall 712 may be positioned on a side of the wall 712 opposite the first portion of the wall 712. The first clip 705 may be couplable to the second clip 706 to fix the first clip 705 and the second clip 706 to the wall 712. The wall 712 may include a first wall 716. The first wall 716 may include the first portion of the wall. The wall 712 may further include a second wall 717 hermetically coupled to the first wall 716. The second wall 717 may include the second portion of the wall 712. The hermetic coupling may at least partially define the compartment 711 between the first wall 716 and the second wall 717. The hermetic coupling may further at least partially define a rim 718 extending from the compartment 711. The coupling between the first clip 705 and the second clip 706 may be coupled by applying pressure to opposing sides of the rim 718. A first portion of the rim may extend along a length of a first side of the package 710 on a first side of the compartment 711. A second portion of the rim may extend along a length of a second side of the package 710 on a second side of the compartment 711. The first side of the compartment 711 may be an opposite side of the compartment 711 relative to the second side of the compartment 711. A first portion of the first clip 705 on the first side of the compartment 711 and a first portion of the second clip 706 on the first side of the compartment 711 may both engage the first portion of the rim. A second portion of the first clip 705 on the second side of the compartment 711 and a second portion of the second clip 706 on the second side of the compartment 711 may both engage the second portion of the rim.

In an implementation, a frame coupling portion 703 may couple the first portion of the first clip 705 to the second portion of the first clip 705. A second frame coupling portion 707 may couple the first portion of the first clip 705 to the second portion of the first clip 705. In an implementation, the frame coupling portion 703 may include a protrusion, and the insert coupling portion 723 may include a groove adapted to conformally receive the protrusion. In an implementation, one or more of the frame coupling portion 703 or the insert coupling portion 723 may include or be an arcuate shape. As used herein, arcuate shapes may include arch-shaped elements, u-shaped elements, or v-shaped elements.

The null fitment 708 may include a groove that accepts a second frame coupling portion 707 of a second clip 706 to secure the null fitment in place. The null fitment 708 serves to provide support to the package 710 in single package configurations of the package arrangement 700. In dual-package configurations, two packages abut each other and are held against each other by clips 705 and 706 of the rigid frame 701. In an implementation, the null fitment 708 may be coupled to the wall 712 by pressure exerted by the second clip 706 from the coupling between the first clip 705 and the second clip 706.

FIG. 8 additionally shows a detail of a fluid transport device 730, comprising an administration device 736 such as a needle or other applicator or a connector configured to mate with a medical device connector such as a Luer lock connector. The fluid transport device 730 further comprises a hub 737.

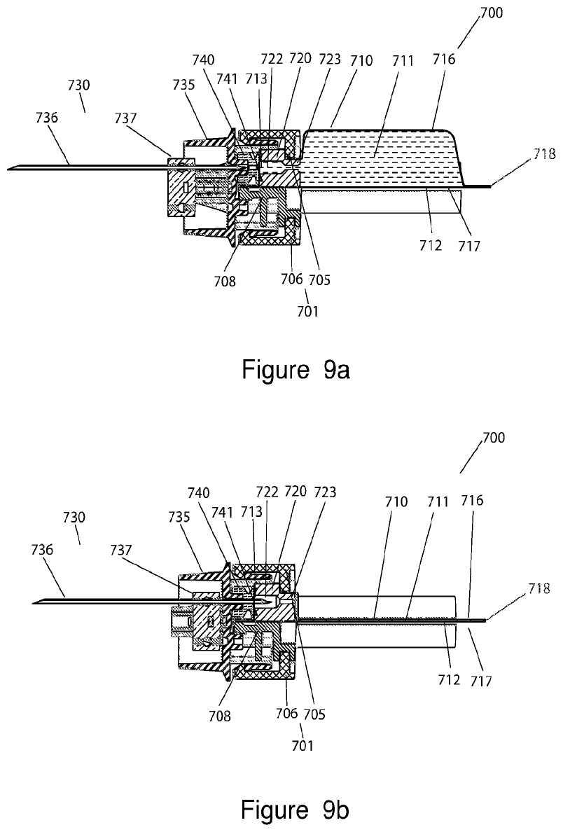

FIGS. 9a-9b illustrate cross-sectional views of an example package arrangement configured for use in an auto-injector device. Specifically, FIG. 9a illustrates a longitudinal section of an example of a package arrangement 700 in a first configuration (e.g., a storage configuration or a sealed configuration). The package arrangement 700 may include a rigid frame 701 comprising a first clip 705 and a second clip 706, a package 710, a rigid insert 720, a fluid transport device 730, an attachment device 735, a septum 740, and a null fitment 708. The package may include the wall 712 which may include a first wall 716, a second wall 717, and a rim 718, the rim 718 formed by joining the first wall to the second wall in fluid-tight or hermetically sealed fashion along their respective perimeters. The beneficial agent storage volume within the compartment 711 may be disposed between boundaries of the wall 712. The boundary of the volume, as shown, may be defined by the first wall 716, the second wall 717, and the rim 718. The insert coupling portion 723 in the rigid insert 720 may be configured to accept a protrusion of the first clip 705 and thereby hold the package 710 steady in the package arrangement subassembly. A conduit extending from the opening 722 formed in the rigid insert 720 can interface with a communication region 713 of the package.

The illustrated example is a single-package embodiment of the package arrangement 700, wherein the null fitment 708 is mounted in the rigid frame 701 in place of a second package. The attachment device 735 comprises an opening confronting the septum 740 that serves to guide the administration device 736 through the septum 740 and the communication region 713. The hub 737 serves as a holder for the administration device 736. The attachment device 735 comprises rib guides that the hub 737 slides against and that direct its axial movement towards the package 710. In an implementation, the hub 737 may include an administration device 736 (e.g., a needle, as illustrated, or a cannula or other fluid transport device) with a passageway (not illustrated) and may be coupled to the attachment device 735. The hub 737 may be configured to be moved towards the attachment device 735. The movement may cause the administration device 736 to pierce the access region 741 and the wall 712 to transition the package arrangement 700 from a first configuration (e.g., a sealed configuration or a storage configuration) in which the compartment 711 is hermetically sealed from the passageway to a second configuration (e.g., a pierced configuration, a dispensing configuration, a ruptured configuration, or a delivery configuration) in which the compartment 711 is in fluid communication with the passageway.

FIG. 9a illustrates the package arrangement 700 in a first configuration (e.g., a sealed configuration or a storage configuration), wherein the fluid transport device 730 is not in fluid communication with the compartment 711 of the package 710. The access region 741 of the septum 740 and the communication region 713 of the wall 712 are intact.

FIG. 9b illustrates the package arrangement 700 in a second configuration (e.g., a pierced configuration, a dispensing configuration, a ruptured configuration, or a delivery configuration) after dispensing of the beneficial agent. The administration device 736 of the fluid transport device 730 has been advanced towards the package 710, and the administration device 736 has penetrated the access region 741 of the septum 740 as well as the communication region 713 of the wall 712, establishing fluid communication with the opening 722 of the rigid insert 720, and by extension, with the compartment 711 of the package 710. The figure shows the compartment 711 in a depleted state, the beneficial agent having been dispensed through the fluid transport device 730.

In an implementation of the package arrangement 700 of one or more of FIG. 7a-7b, 8, or 9a-9b, the rigid frame 701 may include the first clip 705 configured to engage a first portion of the wall 712. The first clip 705 may include the frame coupling portion 703. The second clip 706 may be configured to engage a second portion of the wall 712 on a side of the wall 712 opposite first portion. The first clip 705 may be couplable to the second clip 706 to fix the first clip 705 and the second clip 706 to the wall 712.

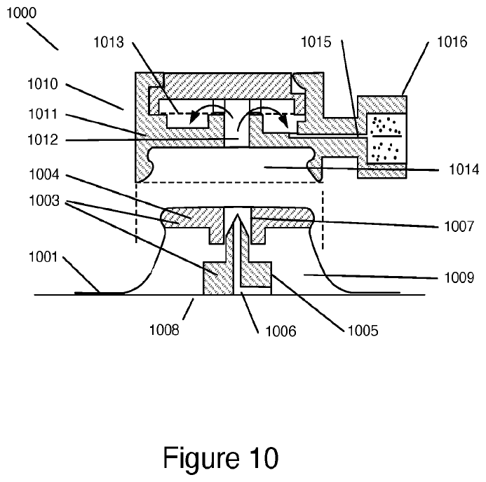

FIG. 10 illustrates a cross-sectional view of an example of a package 1000 comprising a first wall 1001 and a second wall 1008 joined therebetween to form a compartment 1009. The first wall 1001 may include a flexible molded or formed wall. In implementations, the first wall 1001 includes a recess. The second wall 1008 may be an opposing wall that opposes the first wall 1001. In implementations, the second wall 1008 is a substantially flat wall and/or may include a lidding film. The compartment 1009 may contain a beneficial agent or a constituent thereof, or it may be in fluid communication (or communicable) with another compartment or other source of such substance. An insert 1003 is disposed in the compartment and comprises a flange 1004 and a piercing member 1005. The insert 1003 is configured to pierce the first wall 1001 and to establish a fluid-tight seal between the first wall 1001 and a connector 1010. The flange 1004 has an opening 1007 to receive the piercing member 1005. The piercing member 1005 comprises a fluid passageway 1006. The package 1000 may be produced on a Form-Fill-Seal machine, and the production may include the steps of a) feeding the first wall from a roll, b) forming a cavity of a compartment 1009, c) placing the insert 1003 in the cavity of the compartment 1009, d) joining the connector 1010 to the external side of the first wall 1001, e) feeding the second wall 1008 from a roll, f) welding the second wall 1008 to the first wall 1001, and g) cutting the package 1000 of the continuous web strip.

The first wall may be made from a film, in which case forming a cavity of the compartment 1009 may comprise thermoforming, vacuum forming, lug-assist forming, and pressure forming. The first wall may be made from a foil in which case forming the cavity of the compartment 1009 may comprise cold forming. The insert 1003 components, including the flange 1004 and/or the piercing member 1005, may be joined via a press fit of the piercing member 1005 into the opening 1007 of the flange 1004. In one implementation, the flange 1004 and the piercing member may be manufactured as a single component (for example, by injection molding) with a loose connection between the two to allow them to break loose from one another when sufficient force is applied. The flange 1004 may be joined to the first wall via a weld, such as heat stake welding, ultrasonic welding, RF welding, laser welding, or other joining methods known in the art.

The connector 1010 comprises a connector body 1011 comprising a receptacle 1014 configured to receive the first wall 1001 and the flange 1004 to establish a fluid-tight seal between the connector 1010 and the first wall 1001; an inlet 1012 (e.g., an inlet fluid passageway), and an outlet 1015 (e.g., a fluid passageway) leading to an outlet port 1016. A filter 1013 is mounted. A filter 1013 is disposed in the fluid passageway between the inlet 1012 and the outlet 1015. The arrows indicate the beneficial agent's flow path from the inlet 1012 to the outlet 1015. The filter may be designed to prevent particulates from reaching the outlet 1015.

The outlet port 1016 can be an infusion spike port, a connector, a Luer connector, a needle-free connector, a Closed System Transfer Device Connector (CSTD), a dispensing tip, a cannula, a needle, or any other ports known in the art. In one implementation, the connector body 1011 is made from a more compliant/elastic material than the flange 1004 to enhance the seal. For instance, the connector body 1011 can be made from LDPE, EVA or a blend of the two, and the flange 1004 may be made from PET-G or PP.

The piercing member 1005 can move relative to the flange such that the piercing tip of the piercing member 1005 extends through the opening 1007 of the flange 1004 to pierce the first wall 1001 and establish fluid communication between the cavity of the compartment 1009 and the connector 1010. In one embodiment, after piercing, the piercing member 1005 forms a fluid-tight seal with at least one of the flange 1004 and the inlet 1012. The piercing of the first wall may occur when the cavity of the compartment 1009 (or another element of the compartment) is depressed. This can occur in-process during manufacturing or to a finished product by a user.

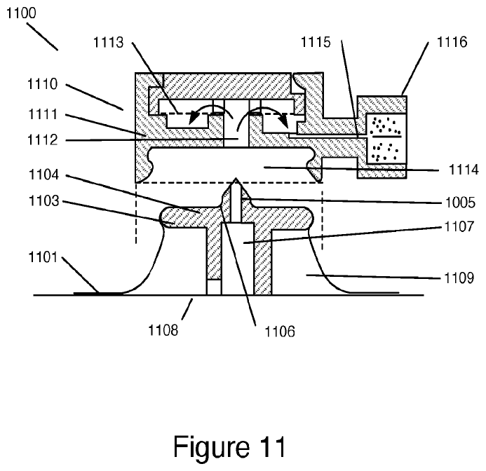

FIG. 11 illustrates a package 1100 mostly similar to package 1000 of FIG. 10, with the exception that the insert 1103 comprises a flange 1104 and a piercing member 1105 rigidly extending from the flange 1104 and passes through the first wall 1101. The package 1100 may recite features similar to the package 1000 of FIG. 10, and like-numbered and/or like-named features of the package 1100 may be arranged and may function similarly to the features of the package arrangement 1000, except as otherwise specified. The opening 2206 in the first wall 1101 can be made by the piercing member 1105 or at a prior step before the insert 1103 is placed in the cavity 1109. A flare may be formed around the opening 1106 surrounding the piercing member 1105. The piercing member 1105 may form a fluid-tight seal with the inlet passageway 1112, through direct contact or through the flare of the first wall 1101. Other details of the description of package 1000 of FIG. 10 may apply to package 1100.

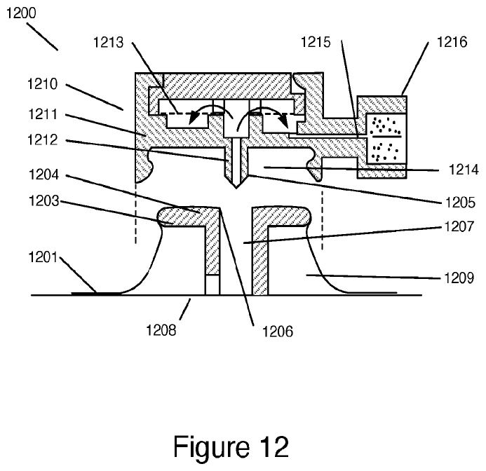

FIG. 12 illustrates a package 1200, which is generally similar to package 1000 of FIG. 10, with the exception that the insert 1203 comprises a flange 1204 and a bore 1207. The package 1200 may recite features similar to the package 1000 of FIG. 10, and like-numbered and/or like-named features of the package 1200 may be arranged and may function similarly to the features of the package arrangement 1000, except as otherwise specified. The connector 1210 comprises a piercing member 1205 in a confronting position to bore 1207. The opening 1206 in a wall 1201 can be made by the piercing member 1205 when mounting the connector 1210 on the insert 1203 or at a prior manufacturing step before mounting the connector 1210. A flair may be formed around the opening 1206 in the wall 1201 surrounding the piercing member 1105. The piercing member 1205 may form a fluid-tight seal with the bore 1207, through direct contact or through the flair of the wall 1201. Fluid communication between the compartment 1209 and the outlet passageway is established through the bore 1207 and the inlet passageway 1212. Other details of the description of package 1000 of FIG. 10 may apply to package 1100.

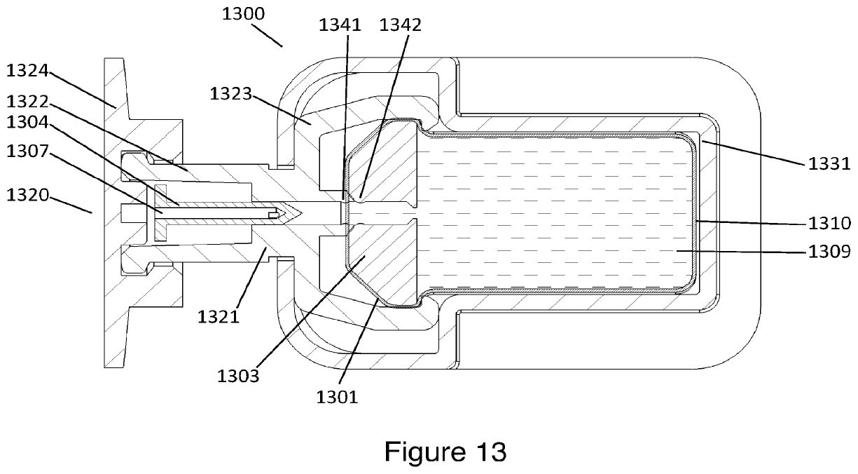

FIG. 13 illustrates a package arrangement 1300 comprising a package 1310, a rigid frame 1331, and a connector 1320 assembly. The package 1310 may include a first wall 1301 forming a compartment 1309 containing a beneficial agent and an insert 1303. The connector may include a body 1321. The body 1321 may include a female Luer connector 1322 and latching arms 1323. The package arrangement 1300 may further include a piercing member 1304 and a Luer cap 1324. The connector 1320 may be secured to the package 1310 by the latching arms 1323 latching to the insert 1303 through the first wall 1301. The arrangement may be such that when the cap is removed and a male Luer connector is mounted on the female Luer connector 1322, the tip of a male Luer connector may advance the piercing member 1307. The advancement may cause the tip to pierce the first wall 1301 and establish fluid communication between the male Luer connector and the compartment 1309. The annular ridges 1341 and 1342 of the connector 1320 and the insert 1303, respectively, may be configured to establish a fluid-tight seal around the piercing member 1307. The flange of the piercing member 1307 may comprise a seal to form a fluid-tight connection with the male Luer tip.

While the invention has been illustrated and described in detail in the drawings and foregoing description, such illustration and description are to be considered as exemplary and not restrictive in character. Accordingly, it should be understood that only the preferred embodiment and variants thereof have been shown and described and that all changes and modifications that come within the spirit of the invention are desired to be protected.

The logical operations making up implementations of the technology described herein may be referred to variously as operations, steps, objects, or modules. Furthermore, it should be understood that logical operations may be performed in any order, adding or omitting operations as desired, regardless of whether operations are labeled or identified as optional, unless explicitly claimed otherwise or a specific order is inherently necessitated by the claim language.

An example package arrangement is provided. The package arrangement includes a package. The package includes a compartment defined by a wall. The wall includes a flexible portion. The compartment is adapted to store a beneficial agent. The compartment is adapted to at least partially collapse in response to applied pressure. The compartment is hermetically sealed in a storage configuration. The package further includes a rigid insert. The rigid insert includes a conduit in fluid communication with the compartment, the rigid insert positioned inside the compartment. The rigid insert includes an insert coupling portion. The package assembly further includes a rigid frame containing at least a portion of the compartment within the rigid frame, the rigid frame including a frame coupling portion. The insert coupling portion is configured to exert pressure on the frame coupling portion through the flexible portion to restrict motion of the rigid insert relative to the rigid frame. The flexible portion hermetically seals a proximal portion of the conduit in the storage configuration.

Another example package arrangement of any preceding arrangement is provided, wherein the insert coupling portion includes one of a recess and a protrusion, and the frame coupling portion includes the other of the recess and the protrusion to couple with the insert coupling portion. The rigid insert includes a proximal portion located proximally relative to the insert coupling portion. The proximal portion having a transverse cross section of a size different from a size of a transverse cross section of the insert coupling portion.

Another example package arrangement of any preceding arrangement is provided, wherein the protrusion protrudes substantially orthogonally to a longitudinal length of the rigid frame.

Another example package arrangement of any preceding arrangement is provided, wherein the hermetic coupling at least partially defines a rim extending away from the compartment, wherein the rigid frame further includes a hollow groove in an interior of the rigid frame, at least a portion of the rim positioned within the hollow groove.

Another example package arrangement of any preceding arrangement is provided, wherein the hollow groove exerts pressure on the rim to reinforce the coupling between a first portion of the wall and a second portion of the wall.

Another example package arrangement of any preceding arrangement is provided, wherein the rigid frame includes a first clip configured to engage a first portion of the wall. The first clip includes the frame coupling portion. The first portion of the wall includes the insert coupling portion. The rigid frame further includes a second clip configured to engage a second portion of the wall. The second portion of the wall on a side of the wall opposite first portion. The first clip is couplable to the second clip to fix the first clip and the second clip to the wall.

Another example package arrangement of any preceding arrangement is provided, wherein the wall includes a first wall. The first wall includes the first portion of the wall. The wall further includes a second wall hermetically coupled to the first wall. The second wall includes the second portion of the wall. The hermetic coupling at least partially defines the compartment between the first wall and the second wall. The hermetic coupling further at least partially defines a rim extending from the compartment. The coupling between the first clip and the second clip is coupled by applying pressure to opposing sides of the rim.

Another example package arrangement of any preceding arrangement is provided, wherein a first portion of the rim extends along a length of a first side of the package on a first side of the compartment, and a second portion of the rim extends along a length of a second side of the package on a second side of the compartment. The first side of the compartment is an opposite side of the compartment relative to the second side of the compartment. A first portion of the first clip on the first side of the compartment and a first portion of the second clip on the first side of the compartment both engage the first portion of the rim. A second portion of the first clip on the second side of the compartment and a second portion of the second clip on the second side of the compartment both engage the second portion of the rim.

Another example package arrangement of any preceding arrangement is provided, wherein the frame coupling portion couples the first portion of the first clip to the second portion of the first clip.

Another example package arrangement of any preceding arrangement is provided, wherein the frame coupling portion includes a protrusion and the insert coupling portion includes a groove adapted to conformally receive the protrusion.

Another example package arrangement of any preceding arrangement is provided, wherein one or more of the frame coupling portion or the insert coupling portion is arcuate.

Another example package arrangement of any preceding arrangement is provided, wherein the rigid frame further includes a null fitment compressed against the wall by the pressure exerted by the second clip from the coupling between the first clip and the second clip.

Another example package arrangement of any preceding arrangement is provided, further includes a second package, the second package compressed against the package by the coupling of the first clip to the second clip.

Another example package arrangement of any preceding arrangement is provided, the rigid frame further including: a septum including an access region pierceable by a fluid transport device and an attachment device couplable to an assembly formed by the coupling of the first clip and the second clip. The attachment device is configured to fix the septum to the wall and orient the access region to coincide with the conduit on opposite sides of the wall in the storage configuration.

Another example package arrangement of any preceding arrangement is provided, further including a hub including a cannula. The cannula includes a passageway. The hub is coupled to the attachment device. The hub is configured to be moved towards the attachment device. The movement causes the cannula to pierce the access region and the wall to transition the package arrangement from the storage configuration in which the compartment is hermetically sealed from the passageway to a delivery configuration in which the compartment is in fluid communication with the passageway.

Another example package arrangement of any preceding arrangement is provided, wherein the wall includes a flexible molded wall including a cavity. The flexible molded wall at least partially defines the flexible portion. The wall further includes an opposing wall. The flexible molded wall is hermetically coupled with the opposing wall to seal the cavity and at least partially define the compartment between the flexible molded wall and the opposing wall. The hermetic coupling at least partially defines a rim extending away from the compartment. At least a portion of the rim is fixed between the first clip and the second clip. The first clip exerts pressure on the second clip through the rim to reinforce the coupling between the flexible molded wall and the opposing wall.

Another example package arrangement of any preceding arrangement is provided, further including a fluid transport device including a passageway. The fluid transport device is configured to pierce the flexible portion. The piercing causes the package arrangement to transition from the storage configuration in which the compartment is hermetically sealed from the passageway to a delivery configuration in which the compartment is in fluid communication with the passageway. The piercing is in a direction substantially orthogonal to a longitudinal length of the package arrangement. The pierced flexible portion is positioned on a side of the package arrangement opposite a side of the packaging arrangement including the insert coupling portion. The conduit includes a substantially right angular bend. The substantially right-angular bend is defined by a first portion of the conduit substantially parallel with a longitudinal length of the package arrangement. The first portion in direct fluid communication with the compartment. The substantially right-angular bend further defined by a second portion of the conduit at least partially orthogonal to a longitudinal length of the first portion. The second portion is in indirect fluid communication with the compartment via the first portion. The second portion is configured to receive at least a portion of the fluid transport device.

Another example package arrangement of any preceding arrangement is provided, further including a cannula. The cannula includes a passageway, a proximal end with a first opening in fluid communication with the passageway, a distal end with a second opening in fluid communication with the passageway, and a hole in a sidewall of the cannula between the proximal end and the distal end. The hole in the sidewall is in fluid communication with the passageway. The conduit includes a first portion substantially parallel to a longitudinal length of the package arrangement. The first portion is in direct fluid communication with the compartment. The conduit further includes a second portion substantially orthogonal to a longitudinal length of the first portion. The second portion is in indirect fluid communication with the compartment via the first portion. The second portion is hermetically sealed by the wall on a first end of the second portion at a first communication region and on a second end of the second portion at a second communication region in the storage configuration. The cannula is configured to transition the package arrangement from the storage configuration in which the compartment is hermetically sealed from the passageway to a delivery configuration in which the passageway is in fluid communication with the compartment by the cannula piercing the first communication region, advancing through the second portion, and piercing the wall at the second communication region. Fluid communication is established between the passageway and the conduit via the hole in the sidewall.

Another example package arrangement of any preceding arrangement is provided, wherein the pierced first communication region and the pierced second communication region maintain a hermetic seal about the cannula in the delivery configuration.

Another example package arrangement of any preceding arrangement is provided, wherein the wall includes a flexible molded wall including a cavity. The flexible molded wall at least partially defines the flexible portion. The wall further includes an opposing wall. The flexible molded wall is hermetically coupled with the flexible molded wall to seal the cavity and at least partially define the compartment between the flexible molded wall and the opposing wall.

Another example package arrangement of any preceding arrangement is provided, wherein the opposing wall includes a lidding film.

Another example package arrangement of any preceding arrangement is provided, further including a fluid transport device including a passageway, The flexible portion includes a communication region. The communication region is configured to be pierced by the fluid transport device at the communication region. The piercing transitions the package arrangement from the storage configuration in which the communication region prevents fluid communication between the passageway and the compartment to a delivery configuration in which the communication region is ruptured by the piercing. The rupture enables fluid communication between the passageway and the compartment via the conduit.

Another example package arrangement of any preceding arrangement is provided, further including a septum. The septum includes an access region pierceable by the fluid transport device during the transition. The access region is positioned to interface with the communication region. The septum is positioned between the package and the fluid transport device.

Another example package arrangement of any preceding arrangement is provided, wherein a portion of the flexible portion of the wall is formed to conform to an interior shape of the rigid frame.

Another example package arrangement of any preceding arrangement is provided, wherein the conduit includes a wide portion with a width wider than a width of a fluid transport device and a narrow portion with a width narrower than the width of the fluid transport device, the narrow portion restricts an extent to which the fluid transport device can penetrate the conduit.

Another example package arrangement of any preceding arrangement is provided, further including a void between the package and the rigid frame and a pressure modification element to control the pressure in the void, the controlled pressure adapted to facilitate expulsion of the beneficial agent from the compartment.

An example package arrangement is provided. The package arrangement includes a compact pre-formed flexible package configured to collapse and expel a stored beneficial agent. The compact, pre-formed flexible package include a compartment adapted to store a beneficial agent and a flexible wall including a communication region. The compartment further includes a rigid insert disposed inside the compartment and confronting the communication region of the flexible wall. The flexible wall includes a first formed side and a second flat lid side joined along a sealed rim.

An example package arrangement is provided. The package arrangement includes a fluid transport device including a passageway. The package arrangement further includes a package. The package includes a flexible molded wall including a recess. The package further includes an opposing wall, the flexible molded wall hermetically coupled with the opposing wall to close the recess and at least partially define a compartment between the flexible molded wall and the opposing wall. The compartment is adapted to store a beneficial agent, the flexible molded wall at least partially collapsible to expel the beneficial agent through the passageway in the second configuration. The package further includes a communication region positioned on at least one of the flexible molded wall and the opposing wall. The communication region is configured to be penetrated by the fluid transport device at the communication region. The penetration transitions the package arrangement from a first configuration in which the communication region prevents fluid communication between the passageway and the compartment to a second configuration in which the communication region is ruptured by the penetration. The rupture enables fluid communication between the passageway and the compartment. The package arrangement further includes a rigid insert including a conduit. The rigid insert is positioned inside the compartment to interface the conduit with an interior surface of the communication region in the first configuration. The conduit is configured to provide fluid communication between the compartment and the passageway in the second configuration. The rigid insert maintains the provided fluid communication when the flexible molded wall collapses.

Another example package arrangement of any preceding arrangement is provided, further including a septum including an access region penetrable by the fluid transport device, the access region positioned to interface with the communication region, the septum positioned between the package and the fluid transport device.

Another example package arrangement of any preceding arrangement is provided wherein gas pressure is used to compress the package to expel the beneficial agent from said package, and wherein the septum prevents the pressure from reaching into the compartment.

Another example package arrangement of any preceding arrangement is provided, further including a second septum, the second septum interfacing with a second communication region, the second communication region positioned on the opposing wall, the septum interfacing with the communication region positioned on the flexible molded wall. Another example package arrangement of any preceding arrangement is provided, further including a rigid frame, the package positioned in an interior of the rigid frame and coupled to the interior of the rigid frame.

Another example package arrangement of any preceding arrangement is provided, wherein the hermetic coupling at least partially defines a rim extending away from the compartment, wherein the rigid frame further includes a lip with a hollow groove, at least a portion of the rim positioned within the hollow groove.

Another example package arrangement of any preceding arrangement is provided, wherein the hollow groove exerts pressure on the rim to reinforce the coupling between the flexible molded wall and the opposing wall.

Another example package arrangement of any preceding arrangement is provided, the rigid insert including a notch configured to receive a protrusion on the interior of the rigid frame and restrict motion of the rigid insert relative to the rigid frame.

Another example package arrangement of any preceding arrangement is provided, the fluid transport device including a septum including an access region penetrable by the fluid transport device, the access region positioned to interface with the communication region. The fluid transport device further includes an administration device, a hub, and an attachment device, the attachment device configured to attach to the rigid frame, securing the septum in a fixed position, and providing a guide for axial movement of the fluid transport device to the access region of the septum.

Another example package arrangement of any preceding arrangement is provided, the fluid transport device including a proximal end configured to dispense the beneficial agent and a distal end configured to receive the beneficial agent into the passageway from the compartment, at least a portion of the distal end configured to penetrate the communication region.

Another example package arrangement of any preceding arrangement is provided, wherein the rigid insert comprises a receptacle configured to receive the distal end of the fluid transport device during the transition.

Another example package arrangement of any preceding arrangement is provided, wherein a proximal end of the passageway is in fluid communication with an administration device for delivering the beneficial agent to a subject, the administration device including at least one of a needle, a micro-needle, a catheter, a tube, a valve, a dispensing tip, or a connector or an adaptor to one of the above.

Another example package arrangement of any preceding arrangement is provided, wherein the access region is adapted to reseal after removal of the dispensing device from the access region.

Another example package arrangement of any preceding arrangement is provided, wherein the communication region is positioned in the opposing wall and wherein the passageway of the fluid transport device is substantially perpendicular to a longitudinal axis of the package.

Another example package arrangement of any preceding arrangement is provided, wherein the rigid insert comprises a receptacle configured to receive the distal end of the fluid transport device in the second configuration, the receptacle at least partially defining the conduit.

Another example package arrangement of any preceding arrangement is provided, wherein a portion of the flexible molded wall is formed to conform to an exterior shape of the rigid insert.

Another example package arrangement of any preceding arrangement is provided, wherein the rigid insert includes a second conduit.

Another example package arrangement of any preceding arrangement is provided, wherein the fluid transport device includes a cannula, the cannula having a proximal end, a distal end, and a hole in a sidewall between the proximal end and the distal end into the passageway.

Another example package arrangement of any preceding arrangement is provided, further including a void between the package and the rigid frame, wherein the package arrangement is adapted to introduce pressurized fluid into a void between the package and the rigid frame to urge the beneficial agent to expel through the passageway.

Another example package arrangement of any preceding arrangement is provided, wherein the opposing wall includes a lidding film.

Another example package arrangement of any preceding arrangement is provided, the package arrangement further including a notch adapted to conform to a contour of a rigid frame.

Another example package arrangement of any preceding arrangement is provided, further including a fluid transport device. The fluid transport device is adapted to pierce a septum and the communication region to establish fluid communication between the compartment and the passageway.

Another example package arrangement of any preceding arrangement is provided, further including a septum disposed on the outer side of the flexible wall of the package confronting the communication region.

Another example package arrangement of any preceding arrangement is provided, wherein the septum establishes a fluid-tight seal that restricts the flow of the beneficial agent from the compartment into the passageway of the fluid transport device and prevents flow of any fluid surrounding the package into said package and into said passageway.

Another example package arrangement of any preceding arrangement is provided, further including a fluid transport device. The fluid transport device includes a distal end configured for access to the package, a proximal end for dispensing the beneficial agent, and a passageway between the distal end and the proximal end.

Another example package arrangement of any preceding arrangement is provided, further including a septum. The septum includes an access region and is configured to direct the fluid transport device into the package.

An example package arrangement for containing and dispensing a liquid beneficial agent is provided. The package arrangement includes a package. The package includes a compartment for holding the liquid beneficial agent. The compartment includes a flexible wall. The flexible wall includes a communication region, an inner side facing the interior of the compartment, and an outer side opposite to the inner side. The package further includes a rigid insert disposed inside the compartment and confronting the communication region of the flexible wall. The package arrangement further includes a fluid transport device. The fluid transport device incudes a distal end configured to access the package, a proximal end for dispensing the beneficial agent, and a passageway between the distal end and the proximal end. The package arrangement further includes a septum disposed on the outer side of the flexible wall of the package, confronting the communication region. The septum includes an access region configured to direct the fluid transport device into the package. The septum establishes a fluid-tight seal that restricts the flow of the beneficial agent from the compartment into the passageway of the fluid transport device and prevents flow of any fluid surrounding the package into said package and into said passageway. The fluid transport device is movable between a first configuration in which the access region and the communication region are not compromised to a second configuration in which the distal end pierces through the access region and the communication region to establish fluid communication between the compartment and the passageway.

Another example package arrangement of any preceding arrangement is provided, wherein the flexible wall further includes a first wall, a second wall, and a flange, the flange formed by joining the first wall to the second wall in fluid-tight fashion along their respective perimeters, the compartment disposed between the first wall and the second wall, the compartment boundary defined by the first wall, the second wall, and the flange.

Another example package arrangement of any preceding arrangement is provided, further including a delivery device configured to couple to the distal end of the fluid transport device

Another example package arrangement of any preceding arrangement is provided, wherein the rigid insert includes a receptacle configured to receive the distal end of the fluid transport device in the second configuration.

Another example package arrangement of any preceding arrangement is provided, wherein the rigid insert includes a conduit.

Another example package arrangement of any preceding arrangement is provided, wherein the distal end of the fluid transport device is an administration device for delivering the beneficial agent to a subject, the administration device including at least one of a needle, a micro-needle, a catheter, a tube, a valve, a dispensing tip, or a connector or an adaptor to one of the above.

Another example package arrangement of any preceding arrangement is provided, wherein gas pressure is used to compress the package to expel the beneficial agent from said package, and wherein the septum prevents the pressure from reaching into the compartment.

Another example package arrangement of any preceding arrangement is provided, wherein the flexible wall is made from a web or a film.

Another example package arrangement of any preceding arrangement is provided, wherein a portion of the flexible wall is formed to accept the shape of the rigid insert.

Another example package arrangement of any preceding arrangement is provided, wherein the access region of the septum is hollow.

Another example package arrangement of any preceding arrangement is provided, wherein the septum is compressed against the rigid insert with the communication region interposed between said septum and said rigid insert, thereby establishing a fluid tight seal between the septum and the outer side of the communication region.

Another example package arrangement of any preceding arrangement is provided, further including a rigid frame surrounding the package. The rigid frame is configured to provide physical support and to couple the package to at least a portion of a delivery device.

Another example package arrangement of any preceding arrangement is provided, wherein the access region of the septum is resealable.

Another example package arrangement of any preceding arrangement is provided, wherein the communication region is disposed in the second wall of the flexible wall and wherein the passageway of the fluid transport device is approximately perpendicular to the long axis of the package.

Another example package arrangement of any preceding arrangement is provided, further including a second septum, wherein the septum confronts the first wall and the second septum confronts the second wall.

Another example package arrangement of any preceding arrangement is provided, wherein the rigid insert includes a second conduit.

Another example package arrangement of any preceding arrangement is provided, wherein the rigid insert includes a notch, and the rigid frame includes an rigid insert configured to accept and mate to a protrusion on the interior of the rigid frame.

Another example package arrangement of any preceding arrangement is provided, wherein the rigid frame includes a lip including a hollow groove configured to accept a portion of the package.

Another example package arrangement of any preceding arrangement is provided, further including a cap, moveably connected to the rigid frame and configured to isolate the proximal end of the fluid transport device from the surrounding environment.

Another example package arrangement of any preceding arrangement is provided, further including a syringe adaptor and a Luer-lock adaptor. The fluid transport device includes a Luer-lock fitment including a sealing septum. The Luer-lock fitment is configured to couple to the syringe adaptor and the Luer-lock adaptor is configured to couple to an intravenous (IV) line.

Another example package arrangement of any preceding arrangement is provided, wherein the rigid frame includes a first clip and a second clip that are joined together to secure the package to the fluid transport device.

Another example package arrangement of any preceding arrangement is provided, wherein the fluid transport device includes an administration device, a hub, and an attachment device, the attachment device configured to attach to the rigid frame, securing the septum in a fixed position, and providing a guide for axial movement of the fluid transport device to the access region of the septum.

Another example package arrangement of any preceding arrangement is provided, wherein the rigid insert includes an engagement surface configured to engage with the attachment device, the attachment device configured to interface the package with a delivery device.

Another example package arrangement of any preceding arrangement is provided, wherein the attachment device is configured to compress the septum against the rigid insert.

Another example package arrangement of any preceding arrangement is provided, further including a null fitment coupled to the second clip and abutting the package

Another example package arrangement of any preceding arrangement is provided, further including a second package, a second septum, and a second fluid transport device

Another example package arrangement of any preceding arrangement is provided, wherein the delivery device is an auto-injector

Another example package arrangement of any preceding arrangement is provided, wherein the fluid transport device includes a cannula. The cannula includes a proximal end, a distal end, a passageway, and a hole in a sidewall between the proximal end and the distal end into the passageway.

Another example package arrangement of any preceding arrangement is provided, wherein a second portion of the cannula resides in a first conduit, the first wall and the second wall are punctured by the cannula, and the hole of the cannula establishes fluid communication between the compartment and the proximal end of the cannula. The first septum and the second septum prevent the beneficial agent from escaping between the cannula and the first wall and between the cannula and the second wall.

Another example package arrangement of any preceding arrangement is provided, wherein pressurized fluid is introduced into a void between the package and the rigid frame to urge the beneficial agent to expel through the cannula.

Another example package arrangement of any preceding arrangement is provided, further including a pump at least partially disposed in the rigid insert and configured to advance the beneficial agent to the proximal end of the cannula.

An example package for containing and transferring a liquid beneficial agent to a fluid transfer device is provided. The package includes a compartment for holding the liquid beneficial agent. The package includes a flexible wall. The flexible wall includes a communication region, an inner side facing the interior of the compartment, and an outer side opposite to the inner side. The package includes a rigid insert disposed inside the compartment and confronting the communication region of the flexible wall. The package includes a rigid frame surrounding the compartment and configured to provide physical support and to couple the package to at least a portion of a delivery device.

Another example package of any preceding package is provided, wherein the flexible wall is made from a web or a film.

Another example package of any preceding package is provided, wherein the flexible wall is formed to accept the shape of the rigid insert.

Another example package of any preceding package is provided, wherein the flexible wall includes a thermoformed layer and a lidding layer.

Another example package of any preceding package is provided, wherein the rigid frame includes snap elements to couple the rigid frame to the at least portion of the delivery device.

While this specification contains many specific implementation details, these should not be construed as limitations on the scope of any technologies or of what may be claimed, but rather as descriptions of features specific to particular implementations of the particular described technology. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In some cases, the actions recited in the claims can be performed in a different order and still achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products. Thus, particular implementations of the subject matter have been described. Other implementations are within the scope of the following claims. Nevertheless, it will be understood that various modifications can be made without departing from the spirit and scope of the recited claims.

As used herein, terms such as “substantially,” “about,” “approximately,” or other terms of relative degree are interpreted as a person skilled in the art would interpret the terms and/or amount to a magnitude of variability of one or more of 1%, 2%, 3%, 4%, 5%, 6%, 7%, 8%, 9%, 10%, 11%, 12%, 13%, 14%, or 15% of a metric relative to the quantitative or qualitative feature described. For example, a term of relative degree applied to orthogonality suggests an angle may have a magnitude of variability relative to a right angle. When values are presented herein for particular features and/or a magnitude of variability, ranges above, ranges below, and ranges between the values are contemplated.

1. A package arrangement, comprising:
a package including:
a compartment defined by a wall, the wall including a flexible portion, the compartment adapted to store a beneficial agent, the compartment adapted to at least partially collapse in response to applied pressure, wherein the compartment is hermetically sealed in a storage configuration; and
a rigid insert including a conduit in fluid communication with the compartment, the rigid insert positioned inside the compartment, the rigid insert including an insert coupling portion; and
a rigid frame containing at least a portion of the compartment within the rigid frame, the rigid frame including a frame coupling portion,
wherein the insert coupling portion is configured to exert pressure on the frame coupling portion through the flexible portion to restrict motion of the rigid insert relative to the rigid frame, wherein the flexible portion hermetically seals a proximal portion of the conduit in the storage configuration.

2. The package arrangement of claim 1, wherein the insert coupling portion includes one of a recess and a protrusion and the frame coupling portion includes the other of the recess and the protrusion to couple with the insert coupling portion, wherein the rigid insert includes a proximal portion located proximally relative to the insert coupling portion, the proximal portion having a transverse cross section of a size different from a size of a transverse cross section of the insert coupling portion.

3. The package arrangement of claim 2, wherein the protrusion protrudes substantially orthogonally to a longitudinal length of the rigid frame.

4. The package arrangement of claim 1, wherein the hermetic coupling at least partially defines a rim extending away from the compartment, wherein the rigid frame further includes a hollow groove in an interior of the rigid frame, at least a portion of the rim positioned within the hollow groove.

5. (canceled)

6. The package arrangement of claim 1, the rigid frame including:
a first clip configured to engage a first portion of the wall, wherein the first clip includes the frame coupling portion, and the first portion of the wall includes the insert coupling portion; and
a second clip configured to engage a second portion of the wall, the second portion of the wall on a side of the wall opposite first portion,
wherein the first clip is couplable to the second clip to fix the first clip and the second clip to the wall.

7. The package arrangement of claim 6, wherein the wall comprises:
a first wall, the first wall including the first portion of the wall; and
a second wall hermetically coupled to the first wall, the second wall including the second portion of the wall, the hermetic coupling at least partially defining the compartment between the first wall and the second wall, the hermetic coupling further at least partially defining a rim extending from the compartment, the coupling between the first clip and the second clip coupled by applying pressure to opposing sides of the rim.

8. The package arrangement of claim 7, wherein a first portion of the rim extends along a length of a first side of the package on a first side of the compartment and a second portion of the rim extends along a length of a second side of the package on a second side of the compartment, the first side of the compartment being an opposite side of the compartment relative to the second side of the compartment,
wherein a first portion of the first clip on the first side of the compartment and a first portion of the second clip on the first side of the compartment both engage the first portion of the rim, and
wherein a second portion of the first clip on the second side of the compartment and a second portion of the second clip on the second side of the compartment both engage the second portion of the rim.

9. The package arrangement of claim 8, wherein the frame coupling portion couples the first portion of the first clip to the second portion of the first clip.

10. The package arrangement of claim 6, wherein the frame coupling portion includes a protrusion and the insert coupling portion includes a groove adapted to conformally receive the protrusion.

11. (canceled)

12. (canceled)

13. The package arrangement of claim 6, further comprising:
a second package, the second package compressed against the package by the coupling of the first clip to the second clip.

14. The package arrangement of claim 6, the rigid frame further including:
a septum including an access region pierceable by a fluid transport device; and
an attachment device couplable to an assembly formed by the coupling of the first clip and the second clip, the attachment device configured to fix the septum to the wall and orient the access region to coincide with the conduit on opposite sides of the wall in the storage configuration.

15. The package arrangement of claim 14, further comprising:
a hub including a cannula, the cannula including a passageway, the hub coupled to the attachment device,
wherein the hub is configured to be moved towards the attachment device, the movement causing the cannula to pierce the access region and the wall, to transition the package arrangement from the storage configuration in which the compartment is hermetically sealed from the passageway to a delivery configuration in which the compartment is in fluid communication with the passageway.

16. The package arrangement of claim 6, the wall comprising:
a flexible molded wall including a cavity, the flexible molded wall at least partially defining the flexible portion; and
an opposing wall, the flexible molded wall hermetically coupled with the opposing wall to seal the cavity and at least partially define the compartment between the flexible molded wall and the opposing wall,
wherein the hermetic coupling at least partially defines a rim extending away from the compartment, wherein at least a portion of the rim is fixed between the first clip and the second clip, wherein the first clip exerts pressure on the second clip through the rim to reinforce the coupling between the flexible molded wall and the opposing wall.

17. The package arrangement of claim 1, further comprising:
a fluid transport device including a passageway, the fluid transport device configured to pierce the flexible portion, the piercing causing the package arrangement to transition from the storage configuration in which the compartment is hermetically sealed from the passageway to a delivery configuration in which the compartment is in fluid communication with the passageway, the piercing in a direction substantially orthogonal to a longitudinal length of the package arrangement, the pierced flexible portion positioned on a side of the package arrangement opposite a side of the packaging arrangement including the insert coupling portion,
wherein the conduit includes a substantially right angular bend defined by:
a first portion of the conduit substantially parallel with a longitudinal length of the package arrangement, the first portion in direct fluid communication with the compartment; and
a second portion of the conduit at least partially orthogonal to a longitudinal length of the first portion, the second portion in indirect fluid communication with the compartment via the first portion, the second portion configured to receive at least a portion of the fluid transport device.

18. The package arrangement of claim 1, further comprising:
a cannula, the cannula including:
a passageway;
a proximal end with a first opening in fluid communication with the passageway;
a distal end with a second opening in fluid communication with the passageway; and
a hole in a sidewall of the cannula between the proximal end and the distal end, the hole in the sidewall in fluid communication with the passageway,
wherein the conduit includes:
a first portion substantially parallel to a longitudinal length of the package arrangement, the first portion in direct fluid communication with the compartment; and
a second portion substantially orthogonal to a longitudinal length of the first portion, the second portion in indirect fluid communication with the compartment via the first portion, the second portion hermetically sealed by the wall on a first end of the second portion at a first communication region and on a second end of the second portion at a second communication region in the storage configuration,
wherein the cannula is configured to transition the package arrangement from the storage configuration in which the compartment is hermetically sealed from the passageway to a delivery configuration in which the passageway is in fluid communication with the compartment, by the cannula piercing the first communication region, advancing through the second portion, and piercing the wall at the second communication region, wherein fluid communication is established between the passageway and the conduit via the hole in the sidewall.

19. (canceled)

20. The package arrangement of claim 1, the wall comprising:
a flexible molded wall including a cavity, the flexible molded wall at least partially defining the flexible portion; and
an opposing wall, the flexible molded wall hermetically coupled with the flexible molded wall to seal the cavity and at least partially define the compartment between the flexible molded wall and the opposing wall.

21. (canceled)

22. The package arrangement of claim 1, further comprising:
a fluid transport device including a passageway, wherein the flexible portion includes a communication region, the communication region configured to be pierced by the fluid transport device at the communication region, the piercing transitioning the package arrangement from the storage configuration in which the communication region prevents fluid communication between the passageway and the compartment to a delivery configuration in which the communication region is ruptured by the piercing, the rupture enabling fluid communication between the passageway and the compartment via the conduit.

23. The package arrangement of claim 22, further comprising:
a septum including an access region pierceable by the fluid transport device during the transition, the access region positioned to interface with the communication region, the septum positioned between the package and the fluid transport device.

24. (canceled)

25. The package arrangement of claim 1, wherein the conduit includes a wide portion with a width wider than a width of a fluid transport device and a narrow portion with a width narrower than the width of the fluid transport device, the narrow portion restricting an extent to which the fluid transport device can penetrate the conduit.

26. The package arrangement of claim 1, further comprising:
a void between the package and the rigid frame; and
a pressure modification element to control the pressure in the void, the controlled pressure adapted to facilitate expulsion of the beneficial agent from the compartment.
